(19) United States
(12) Patent Application Publication
(10) Pub. No.: US 2025/0226111 A1
(43) Pub. Date: Jul. 10, 2025

(54) HEALTH RISK MONITORING METHOD AND APPARATUS FOR SUBSURFACE SOIL POLLUTION OF IMPERVIOUS SURFACE IN URBAN FACTORIES

(71) Applicants: Nanjing Agricultural University, Nanjing (CN); Nanjing University, Nanjing (CN)

(72) Inventors: Penghui Jiang, Nanjing (CN); Yuqing Wang, Nanjing (CN); Manchun Li, Nanjing (CN)

(21) Appl. No.: 19/089,212

(22) Filed: Mar. 25, 2025

(30) Foreign Application Priority Data
Apr. 18, 2024 (CN) 202410465891.1

(51) Int. Cl. G16H 50/30; G06Q 50/26; G16H 50/50

(52) U.S. Cl. CPC G16H 50/30; G06Q 50/265; G16H 50/50

(57) ABSTRACT

Provided are a health risk monitoring method and apparatus for subsurface soil pollution of impervious surface in urban factories. The method includes the following steps: calculating linkage degree between a target chemical industrial park and the town according to a distance between the target chemical industrial park and each town; and determining a health risk of population affected by the target chemical industrial park by using the linkage degree. The health risk of target population is determined by using the linkage degree, which is helpful for the supervision department to take effective countermeasures in time to ensure public health.

### TECHNICAL FIELD

The present relates to the technical field of health risk monitoring of soil pollution, and in particular to a health risk monitoring method and apparatus for subsurface soil pollution of impervious surface in urban factories.

### BACKGROUND

Pollutants in industrial sites are easy to retain and accumulate in the soil, leading to deterioration of physical and chemical properties of the soil. Some substances with strong mobility and volatility spread into rivers and groundwater by means of diffusion, runoff and sedimentation with soil as the medium. However, river water is the main source of drinking water, the pollutants can directly poison human body through direct water drinking or indirectly through food chain. The pollutants accumulated in the soil and water gradually are accumulated in the open and inclusive material and energy cycle, which poses a potential threat to the water quality of the rivers, food security and residents wellness.

According to the bulletin of soil pollution investigation, the exceedance rates of eight inorganic pollutants, i.e., cadmium, mercury, arsenic, copper, lead, chromium, zinc and nickel, are 7.0%, 1.6%, 2.7%, 2.1%, 1.5%, 1.1%, 0.9% and 4.8%, respectively, and heavy metal pollution has gradually become one of the main types of global environmental pollutants. Heavy metal pollution in the soil has the characteristics of high toxicity, strong concealment, high persistence, wide range of harm, difficult degradation, etc., which usually causes harm to human health through oral ingestion, respiratory inhalation, and dermal contact, and may also threaten human health through food chain transmission and enrichment. Embodiments show that long-term intake of Cd, Cr, As and Hg will damage the digestive system, nervous system and human circulatory system, and excessive Pb, Ni and Zn will affect the functions of kidney, lung, stomach and other organs, which may initiate cardiovascular, coronary heart disease and respiratory diseases and increase the probability of inducing cancer. Many diseases in human body may be induced even if the intake concentration is low, and the risks experienced by people of different ages are quite different. In addition, river pollution is mainly organic pollution, with the main pollutants of ammonia nitrogen, biochemical oxygen demand, permanganate index and volatile phenol. Therefore, it is crucial for soil pollution control, water environment restoration and human health and safety guarantee by mastering the pollution degree of heavy metals in water and soil and the water quality of rivers and carrying out systematic assessment of human health risks.

However, a traditional health risk assessment model with fixed parameters cannot identify high-risk heavy metals more accurately, and probabilistic risk assessment has gradually become a new trend in the field of soil pollution risk assessment.

### SUMMARY

In order to overcome the disadvantages in the prior art, an objective of the present disclosure is to provide a health risk monitoring method and apparatus for subsurface soil pollution of impervious surface in urban factories.

To achieve the objective above, the present disclosure provides the following technical solution:

A health risk monitoring method for subsurface soil pollution of impervious surface in urban factories includes:
calculating linkage degree between a target chemical industrial park and each town according to a distance between the target chemical industrial park and the town;
extracting a town with the linkage degree greater than a preset value, and collecting commuting mobile phone signaling data and population mobility mobile phone signaling data between towns;
constructing a relationship matrix of a population mobility network according to the commuting mobile phone signaling data and the population mobility mobile phone signaling data between towns;
determining population affected by the target chemical industrial park according to the relationship matrix of the population mobility network;
calculating daily average intakes of heavy metals of the population affected by the target chemical industrial park under various exposure routes by a human risk assessment model; and
determining a health risk of the population affected by the target chemical industrial park according to the daily average intake.

Preferably, the calculating linkage degree between a target chemical industrial park and each town according to a distance between the target chemical industrial park and the town includes:
determining the linkage degree between the target chemical industrial park and each town by using a formula as follows:

$${F}_{ij} = \frac{{P}_{i} {P}_{j}}{{d}_{ij}^{2}}$$

where Fᵢⱼ represents linkage degree between an i^{-th} chemical industrial park and a j^{-th} town, Pᵢ is population size of the i^{-th} chemical industrial park, Pⱼ is population size of the j-th town, and dᵢⱼ is a distance between the i^{-th} chemical industrial park and the j^{-th} town. Preferably, the calculating daily average intakes of heavy metals of the population affected by the target chemical industrial park under various exposure routes by a human risk assessment model includes:
calculating daily average intakes of heavy metals of the population affected by the target chemical industrial park by using a formula as follows:

$${ADD}_{ing} = \frac{{C}_{i} \times IngR \times ED \times EF}{BW \times AT} \times 1 {0}^{- 6}$$

$${ADD}_{inh} = \frac{{C}_{i} \times InhR \times ED \times EF}{PEF \times BW \times AT}$$

$${ADD}_{dermal} = \frac{{C}_{i} \times SA \times AF \times ABS \times ED \times EF}{BW \times AT} \times 1 {0}^{- 6}$$

where ADD_{ing}, ADDᵢₙₕ and ADD_{dermal} are daily average intakes of heavy metal elements under the routes of hand-oral ingestion, respiratory inhalation and dermal contact; Cᵢ is a measured content of heavy metal i; IngR is a soil ingestion rate; InhR is a soil particle inhalation rate; ED is exposure duration; EF is an exposure frequency; BW is body weight; AT is average lifetime; PEF is an emission factor; SA is surface area of exposed skin; AF is an attachment factor; and ABS is a dermal absorption factor.

Preferably, the determining a health risk of the population affected by the target chemical industrial park according to the daily average intake includes:
determining the health risk of the population affected by the target chemical industrial park by using a formula as follows:

$$HI = \sum {HQ}_{i} = \sum \frac{{ADD}_{i}}{{RfD}_{i}}$$

$$TCR = \sum {CR}_{i} = \sum {ADD}_{i} \times {SF}_{i}$$

where HI is a total non-carcinogenic health risk, TCR is a total carcinogenic risk; HQᵢ and CRᵢ are a non-carcinogenic health risk and a carcinogenic health risk of the heavy metal i under different exposure routes, respectively; RfDᵢ and SFᵢ represent a reference dose and a slope factor of the heavy metal i under different exposure routes, respectively; and ADDᵢ is a daily average intake of the heavy metal element i under different exposure routes.

The present disclosure further provides a health risk monitoring apparatus for subsurface soil pollution of impervious surface in urban factories, including:
a linkage degree calculating module, configured to calculate linkage degree between a target chemical industrial park and each town according to a distance between the target chemical industrial park and the town;
a data acquisition module, configured to extract a town with the linkage degree greater than a preset value, and collect commuting mobile phone signaling data and population mobility mobile phone signaling data between towns;
a relationship matrix construction module, configured to construct a relationship matrix of a population mobility network according to the commuting mobile phone signaling data and the population mobility mobile phone signaling data between towns;
an affected population determination module, configured to determine population affected by the target chemical industrial park according to the relationship matrix of the population mobility network;
a heavy metal intake determining module, configured to calculate daily average intakes of heavy metals of the population affected by the target chemical industrial park under various exposure routes by a human risk assessment model; and
a health risk assessment module, configured to determining the health risk of the population affected by the target chemical industrial park by using a formula.

The present disclosure further provides an electronic device, including a bus, a transceiver, a memory, a processor, and a computer program stored in the memory and capable of being operated on the processor. The transceiver, the memory and the processor are connected through the bus. The computer program, when executed by the processor, can implement steps in the health risk monitoring method for subsurface soil pollution of impervious surface in urban factories above.

The present disclosure further provides a computer readable storage medium. A computer program is stored on the computer readable storage medium, and the computer program, when executed by a processor, can implement steps in the health risk monitoring method for subsurface soil pollution of impervious surface in urban factories above.

The health risk monitoring method and apparatus for subsurface soil pollution of impervious surface in urban factories provided by the present disclosure have the beneficial effects as follows: compared with the prior art, the population affected by the target chemical industrial park can be accurately determined by analyzing the commuting mobile phone signaling data and population mobility mobile phone signaling data. A human risk assessment model is configured to calculate the daily average intake of heavy metals by the population affected by the target chemical industrial park under various exposure routes, and the effects of different routes on human health are comprehensively considered, which is helpful for the supervision department to take effective countermeasures in time to ensure public health and safety.

### BRIEF DESCRIPTION OF THE DRAWINGS

To describe the technical solutions of the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a flowchart of a health risk monitoring method for subsurface soil pollution of impervious surface in urban factories according to an embodiment;

FIG. 2 is a diagram of geo-accumulation index of heavy metal elements according to an embodiment;

FIG. 3 is a diagram of exceedance points of water quality monitoring according to an embodiment;

FIG. 4 is a probability distribution diagram of soil non-carcinogenic risk according to an embodiment;

FIG. 5 is a diagram showing carcinogenic risk of heavy metals Cd and Pb according to an embodiment;

FIG. 6 is a diagram showing carcinogenic risk of heavy metals Cr and Ni according to an embodiment;

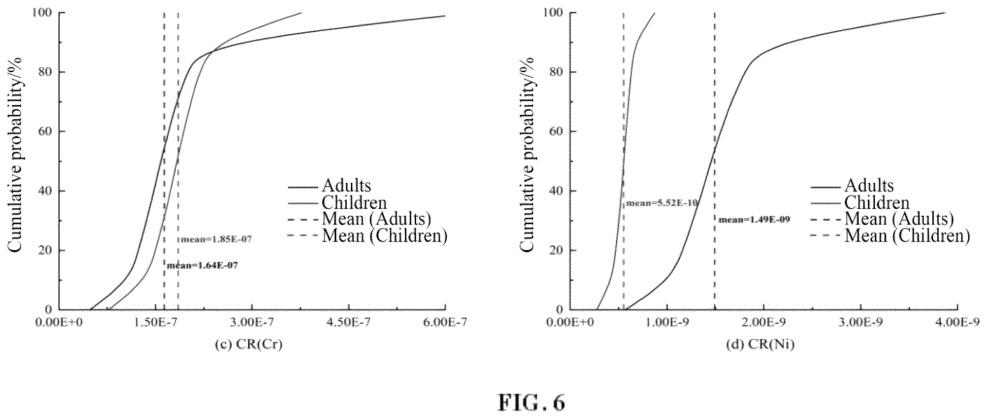

FIG. 7 is a diagram showing carcinogenic risk of a heavy metal As according to an embodiment.

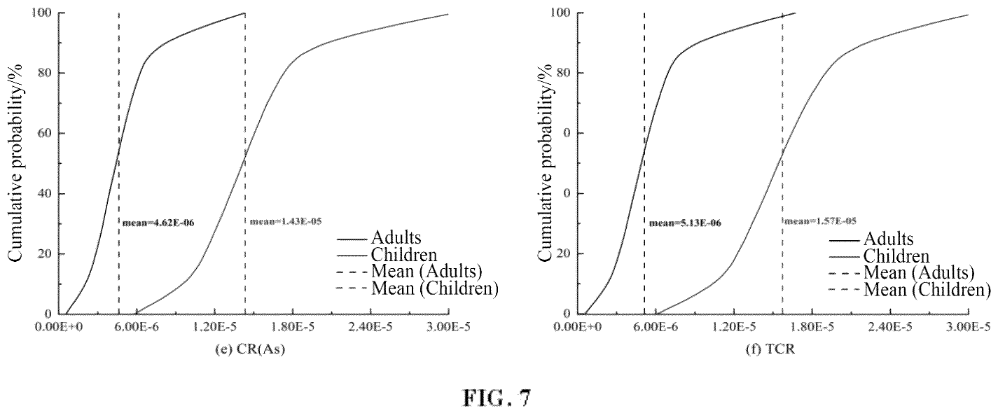

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the scope of protection of the present disclosure.

The term “embodiment” referred to herein means that a particular feature, structure, or characteristic described in conjunction with the embodiment may be included in at least one embodiment of the present disclosure. Such a phrase appearing in various places in the specification does not necessarily refer to the same embodiment, nor does it refer to an independent or alternative embodiment that is mutually exclusive with other embodiments. It is expressly and implicitly understood by those skilled in the art that an embodiment described herein may be combined with other embodiments.

The terms “first”, “second”, “third” and “fourth” in the description and claims of the present disclosure and the accompanying drawings are used to distinguish different objects, rather than describing a specific order. Furthermore, that terms “including” and “have” and any variations thereof are intended to cover non-exclusive inclusion. For example, including a series of steps, processes, methods, etc. is not limited to the listed steps, but optionally includes steps not listed, or optionally includes other step elements inherent to these processes, methods, products, or devices.

In order to make the objectives, features and advantages of the present disclosure more clearly, the present disclosure is further described in detail below with reference to the accompanying drawings and specific embodiments.

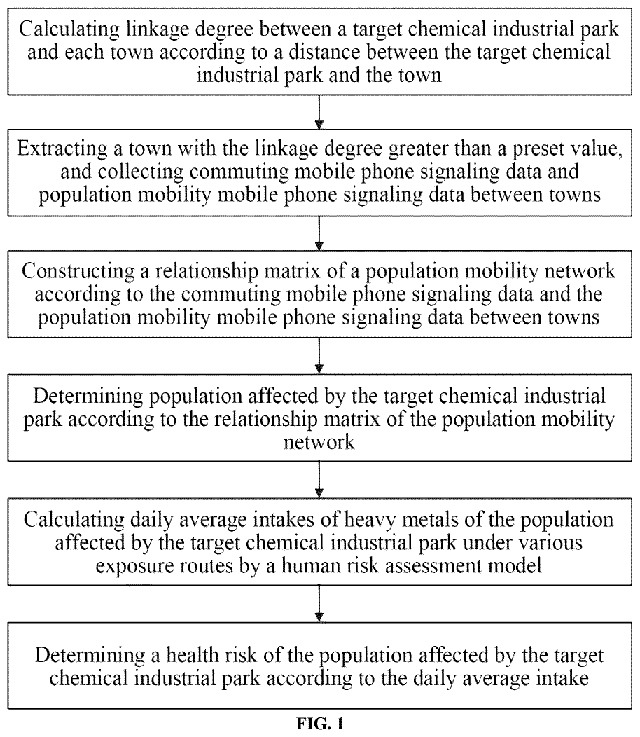

Please referring to FIG. 1, a health risk monitoring method for subsurface soil pollution of impervious surface in urban factories includes:
Step 1: linkage degree between a target chemical industrial park and each town is calculated according to a distance between the target chemical industrial park and the town; in the present disclosure,

$${F}_{ij} = \frac{{P}_{i} {P}_{j}}{{d}_{ij}^{2}}$$

can be used to determine the linkage degree between the target chemical industrial park and the town, where Fᵢⱼ represents linkage degree between an i^{-th} chemical industrial park and a j^{-th} town, Pᵢ is population size of the i^{-th} chemical industrial park, Pⱼ is population size of the j^{-th} town, and dᵢⱼ is a distance between the i^{-th} chemical industrial park and the j^{-th} town;
Step 2: a town with the linkage degree greater than a preset value is extracted, and commuting mobile phone signaling data and population mobility mobile phone signaling data between towns are collected;
Step 3: a relationship matrix of a population mobility network is constructed according to the commuting mobile phone signaling data and the population mobility mobile phone signaling data between towns;
Step 4: population affected by the target chemical industrial park is determined according to the relationship matrix of the population mobility network;
Step 5: daily average intakes of heavy metals of the population affected by the target chemical industrial park under various exposure routes are calculated by a human risk assessment model, where the human risk assessment model is as follows:

$${ADD}_{ing} = \frac{{C}_{i} \times IngR \times ED \times EF}{BW \times AT} \times 1 {0}^{- 6}$$

$${ADD}_{inh} = \frac{{C}_{i} \times InhR \times ED \times EF}{PEF \times BW \times AT}$$

$${ADD}_{dermal} = \frac{{C}_{i} \times SA \times AF \times ABS \times ED \times EF}{BW \times AT} \times 1 {0}^{- 6}$$

where ADD_{ing}, ADDᵢₙₕ and ADD_{dermal} are daily average intakes of heavy metal elements under the routes of hand-oral ingestion, respiratory inhalation and dermal contact; Cᵢ is a measured content of heavy metal i; IngR is a soil ingestion rate; InhR is a soil particle inhalation rate; ED is exposure duration; EF is an exposure frequency; BW is body weight; AT is average lifetime; PEF is an emission factor; SA is surface area of exposed skin; AF is an attachment factor; and ABS is a dermal absorption factor;
Step 6: a health risk of the population affected by the target chemical industrial park is determined according to the daily average intake.

In the present disclosure, the health risk of the population affected by the target chemical industrial park can be determined by using formula:

$$HI = \sum {HQ}_{i} = \sum \frac{{ADD}_{i}}{{RfD}_{i}}$$

$$TCR = \sum {CR}_{i} = \sum {ADD}_{i} \times {SF}_{i}$$

where HI is a total non-carcinogenic health risk, TCR is a total carcinogenic risk; HQᵢ and CRᵢ are a non-carcinogenic health risk and a carcinogenic health risk of the heavy metal i under different exposure routes, respectively; RfDᵢ and SFᵢ represent a reference dose and a slope factor of the heavy metal i under different exposure routes, respectively; and ADDᵢ is a daily average intake of the heavy metal element i under different exposure routes.

The population affected by the target chemical industrial park can be accurately determined by analyzing the commuting mobile phone signaling data and population mobility mobile phone signaling data. A human risk assessment model is configured to calculate the daily average intake of heavy metals by the population affected by the target chemical industrial park under various exposure routes, and the effects of different routes on human health are comprehensively considered, which is helpful for the supervision department to take effective countermeasures in time to ensure public health and safety.

The health risk monitoring method for subsurface soil pollution of impervious surface in urban factories provided by the present disclosure is further described below with reference to specific embodiments.

In this embodiments, 11 chemical companies in a typical urban industrial park are selected as embodiment objects, and the contents of 13 heavy metals such as Zn, Cd, Ba, Pb, Cr, Hg, Ni, Cu and As are determined by using impervious surface mechanical drilling, inductively coupled plasma mass spectrometry, atomic fluorescence spectrometry, etc., and the pollution degree of heavy metals in groundwater and soil is evaluated by single factor pollution index, Nemerow comprehensive index, geo-accumulation index and enrichment factor methods. Five elements, e.g., NH3-N, TP, CODm, VOC and AN, are monitored to analyze the general physical and chemical properties of water quality in surrounding rivers, thus comprehensively exploring the pollution characteristics from two aspects: water body and soil. Then, based on Monte Carlo simulation, the carcinogenic and non-carcinogenic health risks of soil in an embodiment area are assessed by an exposure risk model recommended by US Environmental Protection Agency. On this basis, a directed multi-valued relationship matrix of population mobility in Xinbei District, Changzhou City and Yangtze River Delta are constructed using mobile phone signaling big data and applying a gravity model and complex network analysis methods, the network characteristics of the relationship matrix are measured and analyzed to identify potential health risk population, and summarize the general laws between commuting, population mobility and health risk probability in cities. In this embodiment, it is expected to provide reference for the prevention and control of water and soil environmental pollution, the safety of healthy life of the resident and the formulation of pollution control policies in chemical industrial parks and other cities.

### Source of Big Data

The mobile phone signaling refers to the signal data of auxiliary communication except user data (such as call voice, short message content, internet data packet), which has been widely used in the law of individual travel activities. The mobile phone signaling data used in this embodiment is from a big data platform of China Mobile, and the crawling time interval is from the third quarter of 2020 (Q3) to the first quarter (Q1) of 2023. To carry out gross statistics, spatial distribution and mobility of population based on the mobile phone signaling data, it is necessary to perform pre-processing links such as quality check, de-duplication cleaning and data masking on the original collected signaling data, with specific thoughts as follows: (1) commuting mobile phone data: commuting situation of the population in cities nationwide are monthly counted in the form of district-to-county, the user is counted according to the natural month, a place where the user stays for the longest time from 9 p.m. to 7 a.m. of the next day (and the staying days exceed 15 days) is defined as the residence of the signaling user, a place where the signaling user stays for the longest time from 7 a.m. to 9 p.m. (and the staying days exceed 10 days) is defined as a working place of the signaling user, and the movement of the user from the working place to the residence or from the residence to the working place in two time periods from 6:00 to 9:00 and from 17:00 to 20:00 on weekdays is defined as a commute. Then, de-duplication is performed on the signaling user with the commuting behavior, and a code of a home district/county of a base station is read. Finally, the monthly average commuting volume between districts and counties in China is counted.

Population mobility mobile phone signaling data: the mobility condition of resident population among cities across the country are quarterly counted by taking cities as units. At first, the city where the user stays for the longest resident days is counted as the resident city of the user, which is counted once every natural month. Secondly, the inflow and outflow of population are screened out respectively, grouped monthly and quarterly, and deduplicated to calculate the number of people whose usual residence has changed in the resident population within the city. Finally, top 5 cities with the population inflow and top 5 cites with population outflow of the prefecture-level cities in China are obtained.

### Sample Collection and Detection

According to the requirements of Technical guidelines for environmental site monitoring (HJ25.2-2014) and Technical guideline for investigation and evaluation of soil environment in land for construction, a soil sampling point is set by a professional determining method. The sampling time is from May 12 to Jun. 1, 2018, and a soil sample is collected by mechanical soil holes. When arranging the soil sampling points, a potential pollution area in the site is preliminarily determined according to the plane layout and production technology of each factory, and the depths and quantity of drilling holes are set differently in combination with the topographic characteristics of the site and the site reconnaissance. According to the requirements of the technical guidelines, the sampling positions of surface and deep soil should consider site factors such as contaminant migration, damage of structures and pipelines, and soil characteristics; and in principle, a sampling interval of the soil within a depth of 6 m is 0.5 m, and the sampling interval from 6 m to groundwater aquifer is 1 m. The sampling depth should be deducted from a thickness of non-soil impervious layer on the surface. For soils with different structural characteristics in the vertical direction, the sampling interval in the vertical direction should be appropriately adjusted according to the change of soil structure and the law of contaminant migration. Therefore, in the field investigation and sampling, from the surface, one sample is collected every 0.5 m below the soil within the dept of 6 m. From 6 m to the groundwater aquifer, one sample is collected every 1 m. The specific standards are as follows: a sampling depth of a 3 m soil hole is 3 m below the undisturbed surface, and 6 soil samples are collected at each sampling point, respectively; a sampling depth of a 4.5 m soil hole is 4.5 m below the undisturbed surface, and 9 soil samples are collected at each sampling point; a sampling depth of a 6 m soil hole is 6.0 m below the undisturbed surface, and 12 soil samples are collected at each sampling point, respectively; a sampling depth of a 7.5 m soil hole is 7.5 m below the undisturbed surface, and 15 soil samples are collected at each sampling point, respectively. All soil samples are put into dense bags, respectively. Firstly, the concentration of volatile organic compounds is semi-qualitatively and quantitatively analyzed by a PID detector (photoionization detector). According to readings and pollution signs, two to six soil samples with different sizes are selected from each point and sent to a laboratory for test and analysis. In addition, 11 monitoring wells (all with a depth of 6.0 m) are arranged in Factory 4 to collect soil and groundwater samples, respectively, and the samples are sent to the laboratory for test. The situation of soil sampling and submitted samples in each factory is shown in Table 1.

TABLE 1
Summary table of the number of soil samples
and the number of submitted samples

| | | | Sub-total of | Number of | Number of |
|---|---|---|---|---|---|
| | | Number of | number of | submitted | detected |
| | Layout point | samples | samples | sample | samples |
| Name | (pieces) | (pieces/point) | (pieces) | (piece) | (piece) |
| Factory 1 | 29 | 6-12 | 215 | 102 | 67 |
| Factory 2 | 16 | 9-12 | 162 | 67 | 42 |
| Factory 3 | 46 | 6-12 | 426 | 202 | 106 |
| Factory 4 | 11 | 12 | 121 soil | 132 | 51 soil |
| | | | samples | | samples |
| | | | 11 | | 11 |
| | | | groundwater | | groundwater |
| | | | samples | | samples |
| Factory 5 | 31 | 6-15 | 582 | 254 | 150 |
| Factory 5 | 15 | | | | |
| (Sub-factory 1) | | | | | |
| Factory 5 | 19 | | | | |
| (Sub-factory 2) | | | | | |
| Factory 6 | 38 | 6-12 | 310 | 136 | 90 |
| Factory 7 | 22 | 9-12 | 225 | 110 | 61 |
| Factory 8 | 13 | 9-12 | 129 | 59 | 30 |
| Factory 9 | 23 | 6-12 | 183 | 183 | 63 |
| Factory 10 | 10 | 6-12 | 66 | 66 | 60 |
| Factory 11 | 21 | 6-12 | 204 | 93 | 53 |

In the laboratory sample detection, according to the production situation of the factory, all raw and auxiliary materials and finished products involved in the production process of its products are analyzed to determine the characteristic pollutants. To understand the site pollution in a targeted and comprehensive manner, experimental methods, such as inductively coupled plasma mass spectrometry and atomic fluorescence spectrometry, are used to test chemical elements. In Factory 4, only the PH and total arsenic in soil and groundwater samples are detected, while in other all soil samples, 13 heavy metals are detected and analyzing, including zinc, cadmium, cobalt, lead, chromium, mercury, nickel, copper, arsenic, antimony, silver, beryllium, and tin. In the site environmental site investigation, the soil contrast citation gives priority to the second-class land screening value standard in Risk control standard for soil contamination of development land (GB 36600-2018), if the standard value is vacant, the screening level for non-sensitive land in Screening levels for soil environment health risk assessment of sites in Shanghai, the screening level for industrial land in Beijing Local standard-Screening levels for soil environment health risk assessment of sites, and the screening level for industrial land in US Environmental Protection Agency generic soil screening levels are used in turn. The groundwater evaluation standard gives priority to Class IV standard in Standard for groundwater quality (GB/T 14848-2017). In addition, according to the water quality evaluation requirements of Environmental Quality Standard for Surface Water (GB3838-2002), the conventional physical and chemical indexes are selected as NH₃—N, TP, CODm, VOC and An. The water quality of No.9 Bridge of Zaogang River and Panqiao Bridge of Desheng River East is monitored, and a water sample analysis method refers to The water and wastewater monitoring analysis method.

### Exposure Risk Assessment

A HRA model is configured to calculate daily average intakes of heavy metals in soil by an adult and a child under three exposure routes, with calculating formula respectively as follows:

$${ADD}_{ing} = \frac{{C}_{i} \times IngR \times ED \times EF}{BW \times AT} \times 1 {0}^{- 6}$$

$${ADD}_{inh} = \frac{{C}_{i} \times InhR \times ED \times EF}{PEF \times BW \times AT}$$

$${ADD}_{dermal} = \frac{{C}_{i} \times SA \times AF \times ABS \times ED \times EF}{BW \times AT} \times 1 {0}^{- 6}$$

where ADD_{ing}, ADDᵢₙₕ and ADD_{dermal} are daily average intakes [mg/(kg·d)] of heavy metal elements under the routes of hand-oral ingestion, respiratory inhalation and dermal contact; and the meaning and values of other parameters are shown in Table 2.

TABLE 2
Distribution table of exposure risk assessment parameters
of heavy metals in soil based on Monte Carlo simulation

| | | | Probability | Value | |
|---|---|---|---|---|---|
| Parameter | Symbol | Unit | distribution | Adults | Children |
| Soil particle | IngR | mg/day | Triangular | 4, 30, | 66, 103, |
| intake rate | | | distribution | and 52 | and 161 |
| Soil particle | InhR | m³/day | Point | 19 | 8.6 |
| inhalation rate | | | distribution | | |
| Exposure | ED | year | Point | 24 | 4 |
| duration | | | distribution | | |
| Exposure | EF | day/year | Triangular | 180, 345, and 365 | |
| frequency | | | distribution | | |
| Body weight | BW | kg | Logarithmic | 56.4 | 16.68 |
| | | | distribution | and 11.9 | and 1.48 |
| Average | AT | day | Point | 27740 | |
| exposure time | (carcinogenic) | | distribution | | |
| | AT (non- | day | Point | 8760 | 2190 |
| | carcinogenic) | | distribution | | |
| Skin area | SA | day | Point | 0.54 | 0.23 |
| exposed | | | distribution | | |
| Skin | AF | cm² | Lognormal | 0.49 and | 0.65 and |
| attachment | | | | 0.54 | 1.2 |
| factor | | | | | |

| | | | | |
|---|---|---|---|---|
| Particulate | PEF | mg/cm² | Point | 1.36 × 10⁹ |
| matter release | | | distribution | |
| factor | | | | |
| Dermal | ABS | — | Point | Carcinogenic: |
| absorption | | | distribution | 0.01 |
| factor | | | | Non- |
| | | | | carcinogenic: |
| | | | | 0.001 |

In Table 2, a mean and a standard deviation are used for the lognormal distribution, and a low value and a high value are used for the triangular distribution.

### Healthy Risk Assessment

Limited by the acquisition of exposure parameters, this embodiment only explores the possible non-carcinogenic risks of eight elements in soil to human body, i.e., Zn, Cd, Pb, Cr, Hg, Ni, Cu and As, among which Cd, Pb, Cr, Ni and As also have carcinogenic risks, and the non-carcinogenic and carcinogenic risks possibly caused by heavy metal elements can be obtained by the following calculation formula:

$$HI = \sum {HQ}_{i} = \sum \frac{{ADD}_{i}}{{RfD}_{i}}$$

$$TCR = \sum {CR}_{i} = \sum {ADD}_{i} \times {SF}_{i}$$

in the formula, HI and TCR are a total non-carcinogenic health risk and a total carcinogenic risk, respectively; HQᵢ and CRᵢ represent a non-carcinogenic health risk and a carcinogenic health risk (dimensionless) of the heavy metal i under different exposure routes, respectively; RfDᵢ and SFᵢ represent a reference dose [mg/(kg·d)] and a slope factor [(kg·d)/mg] of the heavy metal i under different exposure routes, respectively, specific values may refer to Table 3.

According to the U.S. Environmental Protection Agency and related research, if HQ/HQᵢ≤1, it is indicated that the heavy metal in the soil has no carcinogenic risk to human body. In contrast, if HQ/HQᵢ>1, it is indicated that that the heavy metal in the soil has carcinogenic risk to human body, and the probability is positively correlated with HI value. When TCR/CRᵢ≤1×10⁻⁶, it is indicated that there is no significant impact on individual health, when 1×10⁻⁶≤TCR/CRᵢ≤1×10⁻⁴, it is indicated that there is a tolerable cancer-causing health risk, while TCR/CRᵢ>1×10⁻⁴, it is indicated that indicates that the individual is exposed to a specific environment, and a certain probability of cancer risk will be caused.

TABLE 3
Values of reference dose and slope factor

| | Reference dose [mg/(kg · d) | | | Slope factor [(kg · d)/mg] | | |
|---|---|---|---|---|---|---|
| | | Respi- | | | Respi- | |
| | Oral | ratory | Dermal | Oral | ratory | Dermal |
| Element | intake | intake | intake | intake | intake | intake |
| Zn | 3E−1 | 3E−1 | 6.00E−2 | — | — | — |
| Cd | 3E−1 | 1E−5 | 1.00E−5 | 6.1 | 6.3 | — |
| Pb | 3.5E−3 | 3.52E−3 | 5.25E−4 | 8.5E−3 | — | — |
| Cr | 3E−3 | 2.86E−5 | 6.00E−5 | 8.5E−3 | 42 | 20 |
| Hg | 3E−4 | 8.57E−5 | 2.EE−5 | — | — | — |
| Ni | 2E−2 | 2.06E−2 | 5.40E−3 | — | 0.84 | — |
| Cu | 4E−2 | 4.02E−2 | 1.20E−2 | — | — | — |
| As | 3E−4 | 1.23E−4 | 1.23E−4 | 1.5 | 15.1 | 3.66 |

In Table 3, “-” represents there is no carcinogenic slope factor.

### Construction of Risk Population Mobility Network

The gravity model originated from Newton's law of gravitation, which revealed the interaction between different entities in a geographical space. The gravity model has been widely used in the fields of regional economic relations, population migration, urban interaction, etc. This model can be configured to quantitatively reflect the urban population mobility and reveal the population contact intensity and leading contact direction. Based on this, it is considered that the linkage between the two areas is directly proportional to their respective gross population and inversely proportional to their distance. Therefore, the linkage between the chemical industrial park and the towns is explored, and the risk population that are more vulnerable to chemical water and soil pollution are identified. The specific model is

$${F}_{ij} = \frac{{P}_{i} {P}_{j}}{{d}_{ij}^{2}} ,$$

in the formula, and Fᵢⱼ represents the linkage between the two areas; Pᵢ and Pⱼ are the population sizes of areas i and j, which are represented by the gross population counted according to jobs-housing mobile phone signaling data in this embodiment; and dᵢⱼ is a distance between the two areas.

### Relationship Matrix of Population Mobility Network

A complex network can abstractly represent a large number of complex systems and their interactions in human society and nature. A typical complex network is composed of numerous nodes and their connections. The nodes represent entities in the system, and connections represent the interrelationships between the entities. In the construction of the population mobility network, the method is configured to analyze various network characteristic indexes to reveal the relationship between the nodes, and macro characteristics of the whole complex network. Because the nodes and connections of the inter-city population mobility network are directional, in practical application, different directional multi-valued population mobility network relationship matrices should be constructed according to the differences between the embodiment areas and the embodiment problems.

In this embodiment, firstly, based on the commuting signaling data, O-D commuting data of five municipal districts and one county-level city are constructed, as shown in Table 4. Secondly, the top 5 cities with population inflow or outflow are established by using the searched population mobility mobile phone signaling data, as shown in Table 5. Finally, through the three types of constructed matrices, the population mobility data of the cities are extracted by means of an ArcGIS network analysis tool to establish a directional connection axis to visualize the connection network of each node city in the network, thus identifying the population with high health risk due to heavy metals in the soil and river pollution in the chemical industrial park.

TABLE 4
O-D commuting data from 2020 July to 2023 March

| | | | Monthly average |
|---|---|---|---|
| Time | Place of residence | Working place | commuting volume |
| 202007 | Jintan District | Xinbei District | 7011 |
| 202007 | Jintan District | Zhonglou District | 10777 |
| 202007 | Jintan District | Wujin District | 54572 |
| 202007 | Jintan District | Tianning District | 4597 |
| 202007 | Jintan District | Liyang City | 24129 |
| . . . | . . . | . . . | . . . |
| 202303 | Zhonglou District | Jintan District | 33127 |
| 202303 | Zhonglou District | Xinbei District | 601106 |
| 202303 | Zhonglou District | Wujin District | 2122 |
| 202303 | Zhonglou District | Tianning District | 546487 |
| 202303 | Zhonglou District | Liyang City | 942670 |

TABLE 5
Data of top 5 cites with population inflow of
Changzhou and top 5 cites with population outflow
of Changzhou from 2020 July-2023 March

| | Place of population | Place of population | Floating |
|---|---|---|---|
| Time | outflow | inflow | population |
| 2020 Q3 | Wuxi City | Changzhou city | 1474 |
| 2020 Q3 | Hangzhou City | Changzhou city | 890 |
| 2020 Q3 | Suzhou City | Changzhou city | 848 |
| 2020 Q3 | Taizhou City | Changzhou city | 709 |
| 2020 Q3 | Huzhou City | Changzhou city | 683 |
| . . . | | | |
| 2023 Q1 | Changzhou city | Wuxi City | 55375 |
| 2023 Q1 | Changzhou city | Nanjing City | 51085 |
| 2023 Q1 | Changzhou city | Xuzhou City | 42872 |
| 2023 Q1 | Changzhou city | Zhenjiang City | 36271 |
| 2023 Q1 | Changzhou city | Suzhou city | 36016 |

### Evaluation Results of Geo-Accumulation Index and Enrichment Factor

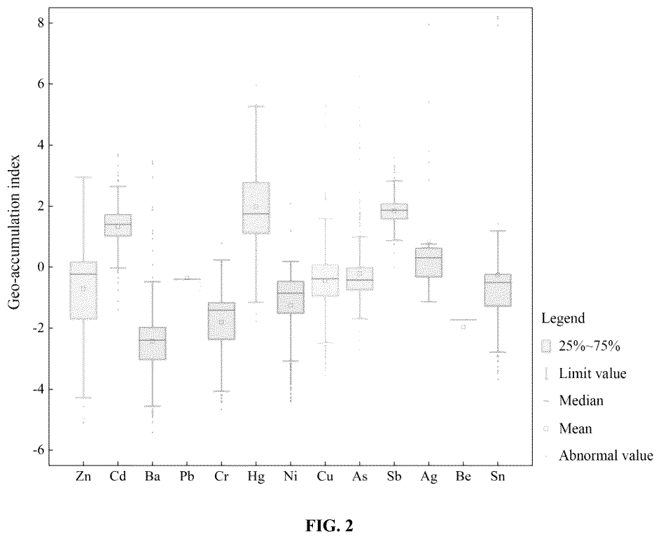

Based on the background values of heavy metal elements in soil in Jiangsu Province, the geo-accumulation index of the heavy metal elements in the embodiment area is calculated. Based on a boxplot of the geo-accumulation index, as shown in FIG. 2, the pollution levels of the 13 heavy metals in the soil are Hg, Sb, CD, Ag, As, Sn, Pb, Cu, Zn, Ni, Cr, Be and Ba in descending order. From the mean, only the three elements (Hg(1.97), Sb(1.84) and Cd(1.33)) have a geo-accumulation index greater than 1, indicating that the cumulative effect is obvious. Igeo value of Hg element fluctuates in the largest range, which spans seven levels from pollution-free to extreme pollution. The pollution levels of Zn, Cd and Sb range from pollution-free and moderate pollution, while the geo-accumulation index values of Ba, Cr, Ni, Ag and Be are all less than 1, showing no pollution or mild pollution. As can be seen from Table 6a that 11.07%, 7.14%, 5.10%, 1.52% and 1.43% of Hg, Ag, Sn, As and Cu are in high pollution level and above, respectively, and the geo-accumulation index values of 2.59% Sn, 1.72% Cd and 0.79% Ba are higher than the moderate pollution level. In addition, to assess the interference degree of human activities on the content of the heavy metals in the soil, enrichment indexes of the 13 elements are calculated, as shown in Table 6b. The result shows that the EF means of the heavy metals Sn, Hg, Sb, Cd, As, Cu, Ag, Zn, Pb, Ni, Ba, Cr and Be in the soil are 79.78, 3.85, 1.74, 1.59, 1.25, 0.85, 0.83, 0.59, 0.51, 0.36, 0.23, 0.23 and 0.7, respectively. The EF means of Cu, Ag, Zn, Pb, Ni, Ba, Cr and Be are all less than 1, indicating that the overall pollution is weak, and Hg, Sb, Cd and As also show different degrees of pollution. However, the EF mean of Sn element far exceeds the threshold value of 40, showing extreme enrichment, revealing that all elements in the chemical industrial park are affected by human activities or natural environment to varying degrees. It can also be obtained that more than 28% of soil sample points in the chemical industrial park are in a medium or higher degree of enrichment, and nearly 2% of sample points are in a very high enrichment or extremely high grade. In addition, the EF values of 69.95% and 53.28% of Sb and Hg respectively exceed the significant enrichment, and the EF values of 31.33% and 17.86% of Cd and Ag are higher than 5, respectively. It shows that more elements of Sb, Hg, Cd and Ag are emitted in the chemical production process in the embodiment area, which leads to a relatively high degree of pollution and a strong risk. The evaluation result is consistent with the geo-accumulation index method.

TABLE 6
Level distribution table of geo-accumulation index (a) and enrichment factor index (b) of heavy metal soil

| Level | Zn | Cd | Ba | Pb | Cr | Hg | Ni | Cu | As | Sb | Ag | Be | Sn |
|---|---|---|---|---|---|---|---|---|---|---|---|---|---|
| (a) | | | | | | | | | | | | | |
| Non-pollution | 62.72% | 5.15% | 95.76% | 78.11% | 99.28% | 7.38% | 96.04% | 72.40% | 75.76% | 0.52% | 39.29% | 100.00% | 83.67% |
| Mild pollution | 30.82% | 19.74% | 1.18% | 18.03% | 0.72% | 13.52% | 2.88% | 20.43% | 16.06% | 4.15% | 42.86% | 0.00% | 9.69% |
| Mild pollution-mode | 5.02% | 64.81% | 1.97% | 1.72% | 0.00% | 38.52% | 0.72% | 3.58% | 4.85% | 60.62% | 0.00% | 0.00% | 1.53% |
| rate pollution | | | | | | | | | | | | | |
| Mode rate pollution | 1.43% | 8.58% | 0.39% | 2.15% | 0.00% | 19.26% | 0.36% | 2.15% | 0.91% | 32.12% | 3.57% | 0.00% | 0.00% |
| Mode rate pollution- | 0.00% | 1.72% | 0.79% | 0.00% | 0.00% | 10.25% | 0.00% | 0.36% | 0.91% | 2.59% | 7.14% | 0.00% | 0.00% |
| severe pollution | | | | | | | | | | | | | |
| Severe pollution | 0.00% | 0.00% | 0.00% | 0.00% | 0.00% | 8.20% | 0.00% | 0.36% | 0.91% | 0.00% | 0.00% | 0.00% | 0.00% |
| Extreme pollution | 0.00% | 0.00% | 0.00% | 0.00% | 0.00% | 2.87% | 0.00% | 0.72% | 0.61% | 0.00% | 7.14% | 0.00% | 5.10% |
| (b) | | | | | | | | | | | | | |
| No enrichment | 35.48% | 1.72% | 94.09% | 32.62% | 96.77% | 2.46% | 58.99% | 35.48% | 31.82% | 0.00% | 10.71% | 91.00% | 42.35% |
| Light enrichment | 49.82% | 9.01% | 1.97% | 56.22% | 2.87% | 7.79% | 39.93% | 47.31% | 51.21% | 0.52% | 39.29% | 9.00% | 46.43% |
| Mode rate enrichment | 12.90% | 57.94% | 1.57% | 9.01% | 0.36% | 36.48% | 0.72% | 13.62% | 11.82% | 29.53% | 32.14% | 0.00% | 6.12% |
| Significant enrichment | 1.79% | 30.90% | 2.36% | 2.15% | 0.00% | 38.93% | 0.36% | 2.51% | 3.03% | 69.95% | 7.14% | 0.00% | 0.00% |
| Strong enrichment | 0.00% | 0.43% | 0.00% | 0.00% | 0.00% | 9.02% | 0.00% | 0.36% | 1.21% | 0.00% | 3.57% | 0.00% | 0.00% |
| Extreme enrichment | 0.00% | 0.00% | 0.00% | 0.00% | 0.00% | 5.33% | 0.00% | 0.72% | 0.91% | 0.00% | 7.14% | 0.00% | 5.10% |

### Analysis of Conventional Physical and Chemical Properties of River Water Quality

The features of water quality cross-section detection data of two rivers collected from 2017 to 2018 are analyzed, as shown in Table 7. As can be seen from the result that the ranges of six monitoring indexes of Zaogang River are all lower than the corresponding standard values, and the pollution degree of each index is ranked as TP, AN, NH₃—N, CODm and VOC in a descending order. Except for NH₃—N, which is highly variable (CV=110 40%), the coefficients of variation of other indexes are less than 70%, especially PH and AN, their contents change little, and the data distribution is relatively concentrated. The water pollution of Desheng River is serious, only a PH value is between the standard values, and standard-exceeding multiples of the means of NH₃—N, TP and CODm are 0.808, 1.5 and 0.865, respectively.

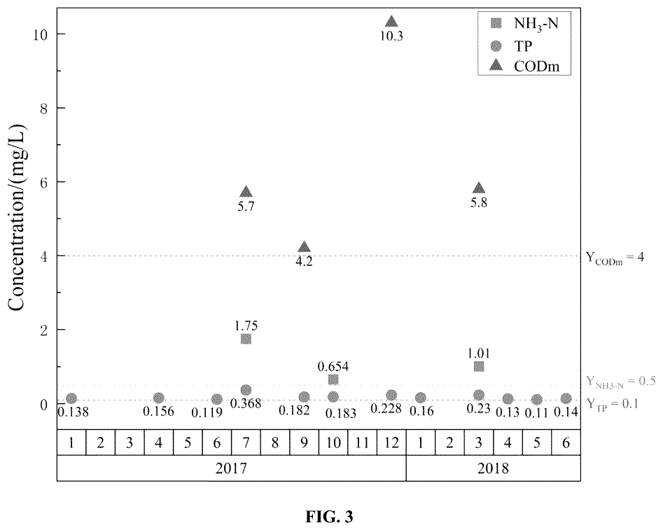

To reflect the elements exceedance conditions at different points of Desheng River in 2017 and 2018, a diagram of corresponding exceedance points is output, as shown in FIG. 3. In the past two years, there are 3 points with NH₃—N exceeding the standard and 4 points with CODm exceeding the standard, and the detection concentration of CODm ranges from 4. 2 to 10. 3, and data is discrete. In 2017 and 2018, the standard-exceeding points of TP accounted for 58.33% and 83.33%, respectively, and the data changed slightly, but was greatly influenced by human activities. In general, the PH values of the two rivers are within the normal range, and the other five indexes all show different degrees of exceeding the standard, the content of chemical component TP is high, which is the most serious pollution to the river water quality.

The reason that the rivers are polluted is largely related to a large number of industrial wastewater and other harmful substances such as heavy metals discharged by factories in industrial parks. In particular, Factory 4 and Factory 6 are close to the river branch, and the industrial sulfuric acid and dimethyl sulfate produced by Factory 4 and Factory 6 are easy to produce acidic wastewater, posing a threat to the surrounding water and soil environment. Moreover, the heavy metal pollution of groundwater and soil then spreads to the whole river channel through diffusion and runoff. Considering that the cross sections of the two rivers are located near towns and villages, the sedimentation and enrichment of heavy metal elements in the soil are more harmful to human health. Meanwhile, as the direct drinking water source of Changzhou people, stricter control measures should be taken to improve the water environmental capacity and water quality.

TABLE 7
Statistical table of water quality characteristics of polluted rivers

| River | Statistical | | Water quality concentration/(mg/L) | | | | |
|---|---|---|---|---|---|---|---|
| name | parameter | PH | NH₃-N | TP | CODm | VOC | AN |
| Gangzao | Minimum value | 7.22 | 0.036 | 0.080 | 1.50 | 0.0005 | 0.0390 |
| River | Maximum value | 7.92 | 1.490 | 0.232 | 5.60 | 0.0028 | 0.0500 |
| | Mean | 7.67 | 0.423 | 0.143 | 2.75 | 0.0014 | 0.0436 |
| | Standard deviation | 0.19 | 0.467 | 0.042 | 1.51 | 0.0009 | 0.0047 |
| | Variable coefficient | 2.48% | 110.40% | 29.37% | 54.91% | 64.29% | 10.78% |
| | Standard value | 6-9 | 1.5 | 0.3 | 10 | 0.01 | 0.1 |
| | Standard-exceeding | \ | 0.282 | 0.477 | 0.275 | 0.140 | 0.436 |
| | multiple | | | | | | |
| Desheng | Minimum value | 7.56 | 0.042 | 0.081 | 1.70 | — | |
| River | Maximum value | 8.04 | 1.750 | 0.368 | 10.30 | | |
| | Mean | 7.81 | 0.404 | 0.150 | 3.46 | | |
| | Standard deviation | 0.140 | 0.410 | 0.071 | 2.10 | | |
| | Variable coefficient | 103.31% | 101.49% | 47.33% | 60.69% | | |
| | Standard value | 6-9 | 0.5 | 0.1 | 4 | | |
| | Standard-exceeding | \ | 0.808 | 1.500 | 0.865 | | |
| | multiple | | | | | | |

In above table, “-” means no data

### Health Risk Assessment of Heavy Metals in Soil

### Non-Carcinogenic Health Risk Assessment

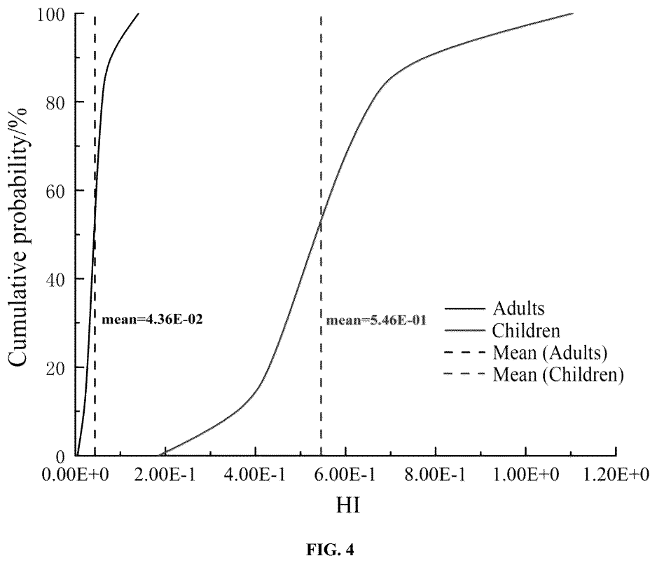

For example, a non-carcinogenic health risk assessment result of heavy metals in the soil in the embodiment is shown in FIG. 4, the non-carcinogenic risks under different exposure routes are obviously different, and the overall performance is that oral intake>respiratory intake>skin intake. The non-carcinogenic risks of heavy metals in the three routes are As>Cr>Pb>Ni>Cu>Hg>Cd>Zn from high to low, and their non-carcinogenic risk indexes HQ are all less than 1. According to the non-carcinogenic health risk index (HI) of different populations, the means of indexes of adults and children are 4.36E-02 and 5.46E-0, respectively. For adults, the non-carcinogenic health risks of heavy metals in the soil can be ignored, but for children, more than 95% of the soil with a risk index ranging from 0.2 to 1, and the soil with a risk index greater than 1 accounts for about 3.4%. Therefore, it can be concluded that the potential non-carcinogenic risk of soil in and around the embodiment area to the public is low. Considering the psychological and physiological characteristics of children, such as finger sucking, outdoor activities and ground contact, children are more likely to unconsciously ingest a certain dose of toxins, and making the non-carcinogenic risk probability higher than that of adults.

### Carcinogenic Health Risk Assessment

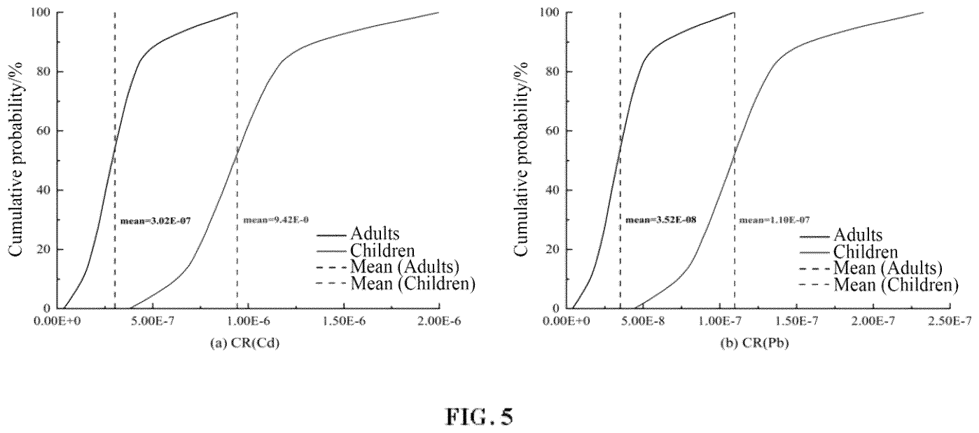

Due to the lack of carcinogenic factors, only the carcinogenic risks and total carcinogenic risks of five metals, i.e., Cd, Pb, Cr, Ni and As, are evaluated, as shown in FIG. 5. The means of carcinogenic risk indexes (CR) of the five heavy metals are ranked as, Cd, Cr, Pb and Ni, among which As, Cd, Cr and Pb have higher carcinogenic risks for children than adults.

In terms of the carcinogenic risk results of individual elements, different from other elements (except Ni) which make the carcinogenic probability of children is always higher than that of adults, in the distribution of cancer probability of the Cr element in the two groups of people shows that the carcinogenic pollution index of adults ranging from 90% percentile to 100% percentile is higher than that of children. The maximum CR values of Pb, Ni and Cr are less than 1.00E-06 in both adults and children, indicating that these three heavy metals are unlikely to cause cancer in the public. It is predicted that the maximum CR value of Cd in adults is 9.39E-07 (≤1.00E-06), while in children, the CR of 70% percentile is equal to 1.05E-06, which exceeds the threshold of cancer risk, indicating that Cd has tolerable carcinogenic risk for children. The CR means of As in adults and children are 4.62E-06 and 1.43E-05, respectively (both greater than 1.00E-06), and their maximum values are both less than 1.00E-04. It can be concluded that As has a relatively high carcinogenic risk to the population, although the carcinogenic risk is still within the tolerable range, it should be paid enough attention to.

The results of TCR assessment show that the heavy metals in the soil of chemical land in the embodiment area have a tolerable carcinogenic health risk to children and adults to some extent. The lifetime cancer risk of the children is higher than that of adults (1.00E-04>1. 57E-05>5. 13E-06), which is related to higher sensitivity to pollutants of the children due to their physiological and behavioral characteristics. As and Cd are identified as the pollutants needing to be preferably controlled in the area, this is because As and Cd are relatively harmful to many system functions of human body, and SF value is relatively large, which makes higher carcinogenic toxicity and cancer risk. This is consistent with the existing embodiments.

### Analysis on Spatial Pattern of Mobile Network of Risk Crowd

The characteristics of commuting are analyzed from the time dimension, the commuting contact in Changzhou in the third and fourth quarters of 2020, the first quarters of 2021, 2022 and 2023 shows the law of “increase-decrease-increase”. The total monthly commuting population in Xinbei District in these four time periods is 2,090,000, 2,227,300, 870,800 and 1,890,800. By analyzing the commuting mobility network among districts and counties in Changzhou, it is concluded that Zhonglou District, Tianning District and Wujin District are closely related to Xinbei District, and the population in the area may be more vulnerable to the harm of water and soil pollution in the chemical industrial park. It can be concluded that commuters in the city are more sensitive to distance. In general, the advantages of commuting distance and commuting mode between cities will increase the frequency and density of commuting, and the corresponding carcinogenic or non-non-carcinogenic risk probability of the individual is increased accordingly.

In this embodiment, the pollution of soil and groundwater in 11 factories is investigated systematically, and the water quality of nearby rivers is monitored. Multiple pollution indexes and health risk assessment models are configured to assess the pollution level of heavy metals, water quality standard-exceeding condition and human health risks in the typical chemical industrial parks. It can be found from the embodiment that chemical plants for producing acidic products such as sulfuric acid and factories for producing pharmaceutical raw materials are more likely to cause heavy metal pollution. Sn, As, Hg, Sb, Cd, Ag in soil samples are more likely to be enriched and accumulated, and excessive levels of NH₃—N, TP and CODm may easily lead to deterioration of water quality. The non-carcinogenic health risk of heavy metals in soil to the public can be ignored (HI<1), and oral intake is the main exposure route, but it has tolerable carcinogenic health risk to the children and adults (1×10⁻⁶≤TCR/CRᵢ≤1×10⁻⁴), and the exposure of children to Cd and As is more harmful. In addition, this embodiment further identifies the risk population that may be affected by chemical production and its pollution, and it is considered that all individuals who have lived or arrived in the park and its surrounding areas may have a certain probability of carcinogenic or non-carcinogenic health risks. In general, the higher the commuting density with a pollution source and the more frequent the population movement, the greater the risk probability of potential carcinogenic or non-carcinogenic risk. According to the results of the embodiment, it is clearly found that there are different levels of pollution in both water and soil. The carcinogenic or non-carcinogenic risks of the population around the example area are within the tolerable range.

The present disclosure provides a health risk monitoring apparatus for subsurface soil pollution of impervious surface in urban factories, including:
a linkage degree calculating module, configured to calculate linkage degree between a target chemical industrial park and each town according to a distance between the target chemical industrial park and the town;
a data acquisition module, configured to extract a town with the linkage degree greater than a preset value, and collect commuting mobile phone signaling data and population mobility mobile phone signaling data between towns;
a relationship matrix construction module, configured to construct a relationship matrix of a population mobility network according to the commuting mobile phone signaling data and the population mobility mobile phone signaling data between towns;
an affected population determination module, configured to determine population affected by the target chemical industrial park according to the relationship matrix of the population mobility network;
a heavy metal intake determining module, configured to calculate daily average intakes of heavy metals of the population affected by the target chemical industrial park under various exposure routes by a human risk assessment model; and
a health risk assessment module, configured to determining the health risk of the population affected by the target chemical industrial park by using a formula.

Compared with the prior art, the beneficial effects of an apparatus for predicting cancer characteristic genes based on molecular evolutionary selective pressure provided by the present disclosure are the same as those of the health risk monitoring method for subsurface soil pollution of impervious surface in urban factories described in the above technical scheme, and thus will not be repeated here.

The present disclosure further provides an electronic device, including a bus, a transceiver, a memory, a processor, and a computer program stored in the memory and capable of being operated on the processor. The transceiver, the memory and the processor are connected through the bus. The computer program, when executed by the processor, can implement steps in the health risk monitoring method for subsurface soil pollution of impervious surface in urban factories above.

Compared with the prior art, the beneficial effects of the electronic device provided by the present disclosure are the same as those of the health risk monitoring method for subsurface soil pollution of impervious surface in urban factories described in the above technical scheme, and thus will not be repeated here.

The present disclosure further provides a computer readable storage medium. A computer program is stored on the computer readable storage medium, and the computer program, when executed by a processor, can implement steps in the health risk monitoring method for subsurface soil pollution of impervious surface in urban factories above.

Compared with the prior art, the beneficial effects of the computer readable storage medium provided by the present disclosure are the same as those of the health risk monitoring method for subsurface soil pollution of impervious surface in urban factories described in the above technical scheme, and thus will not be repeated here.

Specific examples are used herein for illustration of the principles and embodiments of the present disclosure. The description of the embodiments is merely used to help illustrate the method and its core principles of the present disclosure. In addition, a person of ordinary skill in the art can make various modifications in terms of specific embodiments and scope of application in accordance with the teachings of the present disclosure. In conclusion, the content of this specification shall not be construed as a limitation to the present disclosure.

What is claimed is:

1. A health risk monitoring method for subsurface soil pollution of impervious surface in urban factories, comprising:
calculating linkage degree between a target chemical industrial park and each town according to a distance between the target chemical industrial park and the town, wherein the calculating linkage degree between a target chemical industrial park and each town according to a distance between the target chemical industrial park and the town comprises:
determining the linkage degree between the target chemical industrial park and each town by using a formula as follows:
$${F}_{ij} = \frac{{P}_{i} {P}_{j}}{{d}_{ij}^{2}}$$
wherein Fᵢⱼ represents linkage degree between an i^{-th} chemical industrial park and a j^{-th} town, Pᵢ is population size of the i^{-th} chemical industrial park, Pⱼ is population size of the j^{-th} town, and dᵢⱼ is a distance between the i^{-th} chemical industrial park and the j^{-th} town;
extracting a town with the linkage degree greater than a preset value, and collecting commuting mobile phone signaling data and population mobility mobile phone signaling data between towns;
constructing a relationship matrix of a population mobility network according to the commuting mobile phone signaling data and the population mobility mobile phone signaling data between towns;
determining population affected by the target chemical industrial park according to the relationship matrix of the population mobility network;
calculating daily average intakes of heavy metals of the population affected by the target chemical industrial park under various exposure routes by a human risk assessment model,
wherein the calculating daily average intakes of heavy metals of the population affected by the target chemical industrial park under various exposure routes by a human risk assessment model comprises:
calculating daily average intakes of heavy metals of the population affected by the target chemical industrial park by using a formula as follows:
$${ADD}_{ing} = \frac{{C}_{i} \times IngR \times ED \times EF}{BW \times AT} \times 1 {0}^{- 6}$$
$${ADD}_{inh} = \frac{{C}_{i} \times InhR \times ED \times EF}{PEF \times BW \times AT}$$
$${ADD}_{dermal} = \frac{{C}_{i} \times SA \times AF \times ABS \times ED \times EF}{BW \times AT} \times 1 {0}^{- 6}$$
wherein ADD_{ing}, ADDᵢₙₕ and ADD_{dermal} are daily average intakes of heavy metal elements under the routes of hand-oral ingestion, respiratory inhalation and dermal contact; Cᵢ is a measured content of heavy metal i; IngR is a soil ingestion rate; InhR is a soil particle inhalation rate; ED is exposure duration; EF is an exposure frequency; BW is body weight; AT is average lifetime; PEF is an emission factor; SA is surface area of exposed skin; AF is an attachment factor; and ABS is a dermal absorption factor;
determining a health risk of the population affected by the target chemical industrial park according to the daily average intake;
wherein the determining a health risk of the population affected by the target chemical industrial park according to the daily average intake comprises:
determining the health risk of the population affected by the target chemical industrial park by using a formula as follows:
$$HI = \sum {HQ}_{i} = \sum \frac{{ADD}_{i}}{{RfD}_{i}}$$
$$TCR = \sum {CR}_{i} = \sum {ADD}_{i} \times {SF}_{i}$$
wherein HI is a total non-carcinogenic health risk; TCR is a total carcinogenic risk; HQᵢ and CRᵢ are a non-carcinogenic health risk and a carcinogenic health risk of the heavy metal i under different exposure routes, respectively; RfDᵢ and SFᵢ represent a reference dose and a slope factor of the heavy metal i under different exposure routes, respectively; and ADDᵢ is a daily average intake of the heavy metal element under different exposure routes.

2. A health risk monitoring apparatus for subsurface soil pollution of impervious surface in urban factories, comprising:
a linkage degree calculating module, configured to calculate linkage degree between a target chemical industrial park and each town according to a distance between the target chemical industrial park and the town;
wherein the calculating linkage degree between a target chemical industrial park and each town according to a distance between the target chemical industrial park and the town comprises:
determining the linkage degree between the target chemical industrial park and each town by using a formula as follows:
$${F}_{ij} = \frac{{P}_{i} {P}_{j}}{{d}_{ij}^{2}}$$
wherein Fᵢⱼ represents linkage degree between an i^{-th} chemical industrial park and a j^{-th} town, Pᵢ is population size of the i^{-th} chemical industrial park, Pⱼ is population size of the j^{-th} town, and dᵢⱼ is a distance between the i^{-th} chemical industrial park and the j^{-th} town;
a data acquisition module, configured to extract a town with the linkage degree greater than a preset value, and collect commuting mobile phone signaling data and population mobility mobile phone signaling data between towns;
a relationship matrix construction module, configured to construct a relationship matrix of a population mobility network according to the commuting mobile phone signaling data and the population mobility mobile phone signaling data between towns;
an affected population determination module, configured to determine population affected by the target chemical industrial park according to the relationship matrix of the population mobility network;
a heavy metal intake determining module, configured to calculate daily average intakes of heavy metals of the population affected by the target chemical industrial park under various exposure routes by a human risk assessment model,
wherein the calculating daily average intakes of heavy metals of the population affected by the target chemical industrial park under various exposure routes by a human risk assessment model comprises:
calculating daily average intakes of heavy metals of the population affected by the target chemical industrial park by using a formula as follows:
$${ADD}_{ing} = \frac{{C}_{i} \times IngR \times ED \times EF}{BW \times AT} \times 1 {0}^{- 6}$$
$${ADD}_{inh} = \frac{{C}_{i} \times InhR \times ED \times EF}{PEF \times BW \times AT}$$
$${ADD}_{dermal} = \frac{{C}_{i} \times SA \times AF \times ABS \times ED \times EF}{BW \times AT} \times 1 {0}^{- 6}$$
wherein ADD_{ing}, ADDᵢₙₕ and ADD_{dermal} are daily average intakes of heavy metal elements under the routes of hand-oral ingestion, respiratory inhalation and dermal contact; Cᵢ is a measured content of heavy metal i; IngR is a soil ingestion rate; InhR is a soil particle inhalation rate; ED is exposure duration; EF is an exposure frequency; BW is body weight; AT is average lifetime; PEF is an emission factor; SA is surface area of exposed skin; AF is an attachment factor; and ABS is a dermal absorption factor; and
a health risk assessment module, configured to determining the health risk of the population affected by the target chemical industrial park by using a formula as follows:
$$HI = \sum {HQ}_{i} = \sum \frac{{ADD}_{i}}{{RfD}_{i}}$$
$$TCR = \sum {CR}_{i} = \sum {ADD}_{i} \times {SF}_{i}$$
wherein HI is a total non-carcinogenic health risk; TCR is a total carcinogenic risk; HQᵢ and CRᵢ are a non-carcinogenic health risk and a carcinogenic health risk of the heavy metal i under different exposure routes, respectively; RfDᵢ and SFᵢ represent a reference dose and a slope factor of the heavy metal i under different exposure routes, respectively; and ADDᵢ is a daily average intake of the heavy metal element under different exposure routes.

3. An electronic device, comprising a bus, a transceiver, a memory, a processor, and a computer program stored in the memory and capable of being operated on the processor, wherein the transceiver, the memory and the processor are connected through the bus; the computer program, when executed by the processor, is able to implement steps in the health risk monitoring method for subsurface soil pollution of impervious surface in urban factories according to claim 1.

4. A computer readable storage medium, wherein a computer program is stored on the computer readable storage medium, and the computer program, when executed by a processor, is able to implement steps in the health risk monitoring method for subsurface soil pollution of impervious surface in urban factories according to claim 1.
